(19) United States
(12) Patent Application Publication
(10) Pub. No.: US 2005/0191687 A1
(43) Pub. Date: Sep. 1, 2005

(54) Method for multiplexed analyte detection

(76) Inventors: Tianxin Wang, Columbia, MD (US); Xing Xiang Li, Vienna, VA (US)

Correspondence Address: Tianxin Wang, 9768 Early Spring Way, Columbia, MD, 21046, US

(21) Appl. No.: 11/067,237

(22) Filed: Feb. 26, 2005

Related U.S. Application Data
(60) Provisional application No. 60/547,937, filed on Feb. 27, 2004.

(51) Int. Cl. C12Q 1/68; C07H 21/04; C12M 1/34

(52) U.S. Cl. 435/6; 536/24.3; 435/287.2

(57) ABSTRACT

The methods and compositions provided herein are based on use of reporter system to detect multiple analyte in a sample. The reporter system can be a signal amplification system that includes a carrier, typically a particle containing an analyte binding moiety, and multiple copies of a signaling moiety. Different reporter system can bind with different analyte. Different reporter system or their signaling moiety can be distinguished and detected. In various embodiments, the signaling moiety is physically released from its carrier after the carrier has been bound to the analyte and distinguished and detected after the release.

### CROSS-REFERENCE TO RELATED APPLICATION

This application claims priority to U.S. Provisional Application No. 60/547,937 filed on Feb. 27, 2004. The entire disclosure of the prior application is considered to be part of the disclosure of the instant application and is hereby incorporated by reference.

### TECHNICAL FIELD

The methods and compositions provided herein relate to methods for multiplexed analyte detection. The multiplexed analyte detection can utilize a signal amplification system (SAS). The SAS is composed of a structure consisting of (a) multiple signaling moieties (b) a carrier entity or entities and (c) one or more analyte binding moieties.

### BACKGROUND OF THE INVENTION

Detecting biological analytes such as bacteria, antigens, antibodies, receptors, ligands and nucleic acids is pivotal to diagnostic test methods for a wide variety of diseases and conditions and is important to research, forensic and risk assessment applications. Such methods typically rely on specific binding between a target biological analyte and a corresponding analyte binding molecule to form a complex that can be readily detected. For example, bacteria may be detected by binding to antibodies specific for surface antigens on the bacteria. Soluble antigens may be detected by binding to specific antibodies raised against the antigen. Conversely, specific antibodies may be detected by binding to their corresponding antigens (or antigen conjugates). Receptors on cell surfaces indicative of particular cell types may be detected by binding to their corresponding ligands. Conversely, ligands may be detected by binding to their corresponding receptors. Nucleic acids may be detected by hybridizing to substantially complementary nucleic acid sequences. Central to all these detection methods is the ability to detect the formation of a bound complex between the target analyte and the analyte binding molecule, which is distinguishable from non-complexed molecules. Typically the bound complex is detected by one of three basic techniques.

One basic technique for detecting an analyte complex is the ELISA method, which relies on linking an enzyme to an antibody. The enzyme linked species forms a sandwich complex with the analyte and another antibody (or antigen) species typically immobilized on a surface. After washing the surface bound complex to remove unbound enzyme-linked molecules, the bound complex is incubated with a substrate for the enzyme to detect the conversion of the substrate to a product that is measured by conventional spectrophotometeric or chemoluminescent techniques. ELISA methods provide the benefit of relatively high sensitivity, but have the disadvantage of taking a relatively long time to execute to obtain maximum sensitivity. ELISA tests also have other disadvantages such as instability of the linked enzyme, relatively expensive substrates and requiring multiple steps to execute, all of which lead to relatively high costs for ELISA tests.

Another basic analyte complex detection technique is labeling, which relies on detecting a label attached to the analyte binding molecule after it is bound to the analyte. Typically the analyte sample is immobilized on a substrate, incubated with the labeled analyte binding molecule, and then washed to remove unbound labeled molecules. Labeling techniques are most commonly used in nucleic acid detection methods where the analyte binding molecule is a nucleic acid probe that is hybridized to a complementary sequence of the target analyte nucleic acid. A variety of label types have been used in this regard, including for example, radioactive, fluorescent, chemiluminescent and electroluminescent species. A variety of substrates have also been used, from simple filter-like membranes to complex nucleic acid chip arrays. In the clinical area, the most commonly used nucleic acid binding tests are for screening blood for viruses (e.g., HIV, HCV and HBV) or for HIV viral load testing. Viral load tests are used to measure viral concentration in the plasma as a means to monitor effectiveness of anti-viral drug therapy or disease progression. One of the major disadvantages of conventional labeling techniques is that the amount of labeled signal molecules attached to the probe is limited by the size of the probe and the necessity of protecting the binding domains for hybridization. This limits the sensitivity of detection, which is sometimes addressed by analyte amplification techniques such as PCR (polymerase chain reaction) to amplify the target analyte nucleic acid. PCR adds another level of complexity (and variability) associated with the enzymes, reagents and protocols needed for reliable PCR.

Furthermore, techniques for multiplexed analyte detection, that is, simultaneously performing different assays on the same sample within the same reaction vessel, are on the rise. One major technology for multiplexed analyte detection is the microarray platform, e.g. DNA chip and protein chip. Another strategy is using bead-and particle-based multiplexed assays called multiplexed bead-based assays, such as BD Biosciences'Cytometric Bead Array or the xMAP™ technology from Luminex. Besides the biochip method and bead based array mentioned above, several other companies are working on different strategies for multiplexed detection, for example, the BeadArray™ technology from Illumina employs optical fiber and addressable beads that self-assemble into microwells etched into an array substrate, effectively generating the array at run-time. Quantum Dot (QDC) is developing a product line using QDot-labeled beads for multiplexed assays, with applications for SNP detection, immunoassays. Nanoplex Technologies has developed a technology for manufacturing tiny, cylindrical particles that serve as the nanoscale equivalent of conventional bar codes, which can be complexed directly to biological molecules for various applications, including multiplexed assays. PharmaSeq has developed a novel system for multiplexed DNA analyses using light-activated microtransponders. However, these methods require expensive instrumentation and provide unsatisfactory sensitivity in many applications.

Accordingly, there is a need in the art for compositions and methods for improving the sensitivity, speed and simplicity of analyte detection, and especially for such compositions and methods that are readily adaptable for detecting a wide variety of analytes including multiplexed analyte detection.

### SUMMARY OF THE INVENTION

The current invention relates to methods and compositions for multiplexed analyte detection. In one aspect, there are provided compositions for analyzing a sample for the presence of at least one analyte. In various embodiments, there are provided, reporter systems for detecting corresponding analyte in a sample; each reporter system includes an analyte binding moiety and a signaling moiety. Different reporter systems or part of them can be distinguished from each other.

In another aspect, there are provided methods for analyzing a sample for the presence of one or more analyte targets. In some embodiments, the methods comprise contacting the sample with reporter systems to form the bound complex with the corresponding analyte target; removing reporter systems that do not bind the analyte and retaining reporter systems that do bind the analyte; distinguishing the retained reporter systems. In other embodiments, the methods comprise contacting the sample with reporter systems to form the bound complex with the corresponding analyte target and distinguishing the bound reporter systems without removing reporter systems that do not bind the analyte. The signaling moiety of the reporter systems after being released or the whole reporter systems are distinguished based on different physical and/or chemical characteristics of different reporter systems.

In yet another aspect of the invention, a signal amplification system (SAS) is used to increase the sensitivity for analyte detection. The SAS is composed of a structure consisting of (a) multiple signaling moieties (b) a carrier entity or entities and (c) one or more analyte binding moieties.

### BRIEF DESCRIPTION OF THE DRAWINGS

FIG. 1 is a schematic drawing of one specific embodiment of the methods and compositions provided herein, where the signaling moiety 1, e.g., chemiluminescent compound, and the analyte binding moiety 3 are attached to the carrier 2.

FIG. 2 is a schematic drawing of one specific embodiment of the methods and compositions provided herein, where the analyte binding moiety 3 is attached to the signaling moiety 1.

FIG. 3 is a schematic drawing of another specific embodiment of the methods and compositions provided herein, where signaling moiety 1 is attached to the analyte binding moiety 3.

FIG. 4 is a schematic drawing of a preferred embodiment of the methods and compositions provided herein, where the signaling moiety 1, or their derivatives, is encapsulated in microparticles.

FIG. 5 depicts an example of analyte detection using the SAS, where a microwell plate is used as the solid phase substrate.

FIG. 6 depicts another example of analyte detection using the SAS, where the magnetic particles are used as solid phase substrate.

FIG. 7 depicts two fluorescent compounds containing releasable signaling moieties.

FIG. 8 depicts an example of multiplex detection of two analyte using color-coded second analyte binding molecules.

### DETAILED DESCRIPTION OF THE INVENTIONS AND THE PREFERRED EMBODIMENT

Prior to describing various exemplary embodiments in detail, to aid one of ordinary skill in the art in understanding the methods and compositions provided herein, the following terms are defined herein as a reference. The definitions are provided for convenience only, and do not limit the ordinary meaning of the terms as would be understood by one of ordinary skill in the art, unless the definitions provided below conflict with such ordinary meaning, in which case the definition provided herein control.

The terms “analyte binding molecule” and “analyte binding moiety” are used interchangeably to mean a molecular species or part of them having a domain that binds to a desired analyte. Example analyte binding moieties include, but are not limited to, nucleic acids, proteins, peptides, antigens, antibodies, ligands, small molecules, polymers, receptors and the like.

A “carrier” is a soluble or insoluble polymeric species that can be associated with multiple molecular species at multiple sites through covalent or non-covalent bonds, and/or by encapsulation within a matrix. Example natural or synthetic polymers that can act as carriers include, but are not limited to, crystals, beads, aggregates, microspheres, oligomers (such as peptides), linear or cross-linked polymers (such as poly lysine, poly acrylic acid, proteins) or highly branched macromolecules (such as dendrimers).

A “releasable linker” or “releasable linkage” is a linker that connects two species together, and which contains a linkage that can be specifically cleaved or dissociated by the action of an enzyme, a particular chemical species, by light or other physiochemical process that cleaves or dissociate the linker. One example of a releasable linker is formed with a single stranded nucleic acid having both a poly A tail and a target specific sequence. The poly A tail can be hybridized to a first poly T nucleic acid at one end and to the target nucleic acid at the other end. When the hybrid is melted by temperature or pH, the oligonucleotide can be released from the hybrid and bind another poly T sequence or another target sequence.

The term “releasable” as used herein with respect to signaling molecules, linkers, adaptors that are associated with a carrier, means that a molecular species can be liberated from association with the carrier by treating the carrier to a condition particularly for the purpose of releasing the molecular species.

A “releasing condition” is to expose the linker to a physical process or to chemical reagents that will release a particular molecular species from association with another. By way of example, and not by limitation, if the molecular species is non-covalently associated with the carrier by being encapsulated therein, a releasing condition can be dissolving, swelling or crushing the carrier. If the molecular species is associated with the carrier by hybridization between complementary nucleic acids, a releasing condition can be heating the carrier and/or changing the pH to melt the hybrid. If the molecular species is covalently associated with the carrier through a covalent bond, a releasing condition can be treating the carrier to a chemical or physical process designed to cleave the covalent bond, for example, by using light to cleave a photolabile bond, using an enzyme to cleave an enzymatically labile bond, or using a reducing reagent to cleave a disulfide bond.

A “microsphere” is a particle having a largest dimension of 100 millimeters or less.

A “particle” is species of a carrier that is insoluble in an aqueous based solvent.

The terms “signaling molecule” and “signaling moiety” are used interchangeably. A “signaling moiety” is a molecule, or part of a molecule or derivative of a molecule, that contains a species having a specifically detectable physical or chemical property. In addition, a signaling moiety includes a species that is capable of producing a detectable physical or chemical property by interacting with another species. By way of example and not by limitation, typical signaling moieties include chemiluminescent, electrochemiluminescent, fluorescent, chromogenic, electrochemical and radioactive species. In certain embodiments, an enzyme is also a signaling moiety if the enzyme is capable of reacting with a substrate to generate a detectable chemiluminescent, electrochemiluminescent, fluorescent, or chromogenic product. Thus for example a luciferase or peroxidase may be considered a signaling moiety because when placed in sample containing the appropriate substrates, chemiluminescent products will be produced by the enzyme.

A “substrate” is a solid phase material, which can be attached to a molecular species. A particle is one form of a substrate, as are sample wells, test tube walls, microtiter dishes and the like.

The current invention relates to methods and compositions for multiplexed analyte detection. In various embodiments, there are provided reporter systems for detecting corresponding analyte in a sample, each reporter system includes an analyte binding moiety and a signaling moiety. One example of a reporter system is fluorescent group labeled nucleic acid probe or antibody, where the signaling moiety is the fluorescent group and the binding moiety is nucleic acid or antibody. In order to detect multiple species of analyte target, multiple types of reporter system are required. Each type of the reporter system can bind with one of the analyte species specifically. Different reporter systems can be distinguished from each other with suitable analytical methods. In some embodiments, detection of the analytes is normally accomplished via specific binding of reporter systems to a solid substrate such as microwell plate or magnetic particles. After washing away unbound reporter systems, the bound reporter systems can be distinguished and measured. Therefore, the presence of a special reporter system indicates the presence of the corresponding analyte. For example, in order to detect two nucleic acid targets, two fluorescent group labeled nucleic acid probes that can bind with the solid substrate after binding selectively with corresponding nucleic acid targets can be used as two reporter systems. After binding and washing, the bound reporter systems are released from solid substrate for detection. The two fluorescent nucleic acid probes are designed to have different sequence so that they have different retention time and can be distinguished and measured by HPLC (high performance liquid chromatography) or CE (capillary electrophoresis) equipped with fluorescent detector. They can also be distinguished and detected with mass spectrometer if these two probes have different mass. Therefore the presence and a mount of two nucleic acid targets are determined. Alternatively, the signaling moiety of the reporter systems is releasable and different for each reporter system and an additional releasing step is performed to the bound reporter systems. For example, the released signaling moieties (e.g. molecules) from corresponding reporter systems are different in chemical or physical property (e.g. fluorescent molecules having different lipophilicity) and therefore they can be distinguished and detected with reversed phase HPLC or CE (e.g. micellar capillary electrophoresis, MECC or Capillary zone electrophoresis, CZE if they have different charge/mass ratio). In this case, the presence and amount of the special analyte are determined from the detection of corresponding released signaling moieties.

Further more, a signal amplifying system (SAS, the SAS is also described in U.S. Provisional Application Nos. 60/532,088 and 60/532,089 and 60/540,576 and their corresponding PCT application) may be used as the reporter system. The SAS contains three major components, (a) multiple numbers of signaling moieties (b) a carrier entity or containing the signaling moieties, and (c) one or more analyte binding moieties specific for an analyte. The signaling moieties typically can be chemiluminescent compounds, electrochemical compounds, fluorescent compounds, electrochemiluminescent compounds, chromogenic compounds, radioactive compounds or their precursors or enzymes that are capable of generating such compounds as a product of an enzymatic reaction with a suitable substrate. In some embodiments, the binding moiety itself is the carrier entity (e.g. a fluorescent dye labeled antibody).

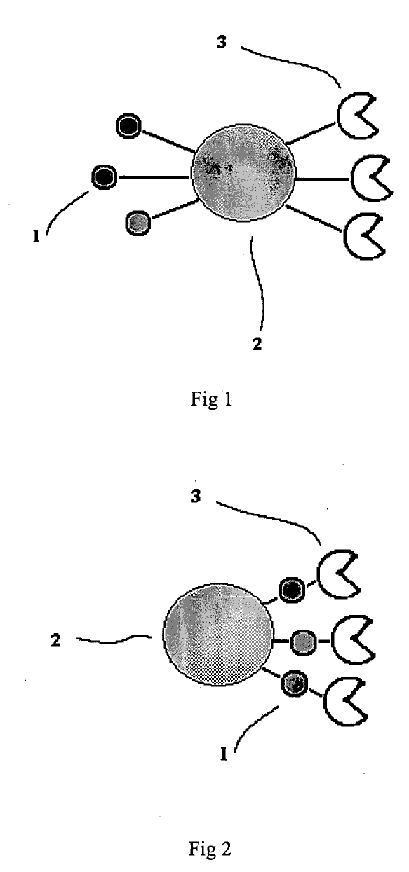

Certain aspects of SAS provided herein may be better understood by referring to FIG. 1. In general, the SAS comprises a large number of signaling moieties such as fluorescent compounds or their derivatives (such as their precursors) 1, which are attached to a carrier entity 2, which also carries one or more analyte binding moieties 3 specific for an analyte or analytes. One SAS unit can have multiple copies of fluorescent molecules and one or more copies of analyte binding moieties. Preferably the number of the signaling moieties in each SAS unit should be the same or similar for sensitive and reproducible detection. In order to specifically detect certain analyte, the analyte binding moieties in the SAS need to have specific affinity for the analyte, or for an adaptor that permits specific binding to the analyte or analytes.

FIG. 2 depicts one specific embodiment, where the analyte binding moiety 3 is attached to the signaling moiety 1 or their derivatives, which are coupled to the carrier 2.

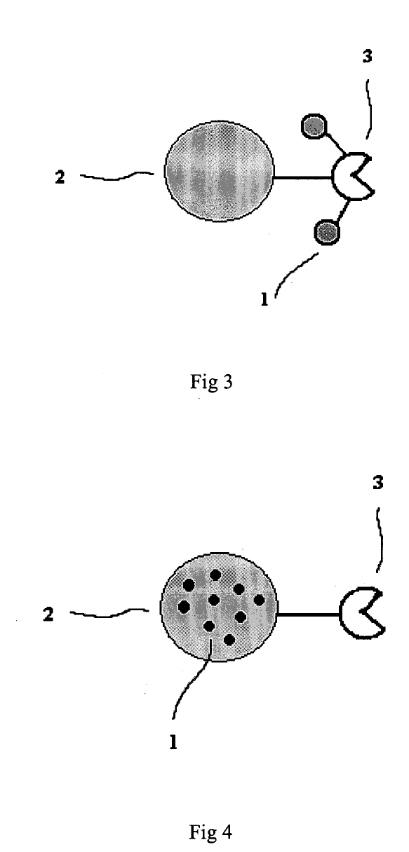

Conversely, as depicted in FIG. 3, the signaling moiety 1 or their derivatives are attached to carrier 2 through the analyte binding moiety 3. The key aspect of these embodiments is that either the analyte binding moiety 3 or the signaling moiety 1, but not both, are directly linked to the carrier.

FIG. 4 illustrates a preferred embodiment of the methods and compositions provided herein, where the signaling moiety 1 or their derivatives are encapsulated in microparticle based carrier 2 and the analyte binding moiety 3 are conjugated on the surface of the microparticles 2. This method allows encapsulation of large number of signaling moieties 1 while large numbers of analyte binding moieties 3 can still be labeled to the particle surface.

Detection of an analyte is normally accomplished via specific binding of reporter system (e.g. SAS) units to a solid substrate such as microwell plate or magnetic particles. After washing away unbound SAS, the bound SAS can be measured for the presence of signaling moieties 1 or after the signaling moieties 1 are released from the carriers. If a chemiluminescent compound is used as the signaling moieties 1, it can be detected using a luminometer or electro-luminometer. The magnitude of amplification is related to the number of signaling moieties on an SAS unit. The more signaling moieties on a carrier entity, the higher the amplification magnitude it can have.

One type of signal moiety is a chemiluminescent moiety. Examples for these types of compounds include both chemiluminescent compound (e.g., acridinium and its derivatives, proteins that can generate light, enzymes that can catalyze chemiluminescence reaction) and electrochemiluminescent agents (e.g., certain organic compounds or rare earth elements in appropriate chelators). Other suitable signaling moieties include, but are not limited to, fluorescent compounds (such as fluorescein), fluorescent quantum dots, or rare earth elements (e.g., Europium in the form of salt, chelate, oxide, metal etc.), electro-chemiluminescent compounds (such as rare earth elements) and dyes. It is understood that signaling moieties also include the precursors, derivatives, activators or inhibitors of signal molecules. It is also understood that suitable signaling moieties include those described in scientific journals and other source of public information.

Appropriate fluorescent groups here includes, but is not limited to, anything that generates fluorescent light signal under appropriate conditions or the precursors or derivatives that gives rise to such compounds. Examples for this type of compounds include both fluorescent compounds, proteins that can generate fluorescent light. A non limiting list is given here: fluorescent squaraine dyes, e.g., red dye which is 1,3-bis[(1,3-dihydro-1,3,3-rimethyl-2H-indol-2-ylidene)methyl]-2,4-dihydroxy-cycl obutenediylium, bis(inner salt); orange dye, e.g. 2-(3,5-dimethylpyrrol-2-yl)-4-(3,5-dimethyl-2H-pyrrol-2-ylidene)-3-hydroxy-2-cyclobuten-1-one; cyclobutenedione derivatives, substituted cephalosporin compounds, fluorinated squaraine compositions, symmetrical and unsymmetrical squaraines, alkylalkoxy squaraines, or squarylium compounds; phthalocyanines and naphthalocyanines; 3-Hydroxypyrene 5,8,10-Tri Sulfonic acid, 5-Hydroxy Tryptamine, 5-Hydroxy Tryptamine (5-HT), Acid Fuhsin, Acridine Orange, Acridine Red, Acridine Yellow, Acriflavin, AFA (Acriflavin Feulgen SITSA), Alizarin Complexon, Alizarin Red, Allophycocyanin, ACMA, Aminoactinomycin D, Aminocoumarin, Anthroyl Stearate, Aryl- or Heteroaryl-substituted Polyolefin, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, BOBO 1, Blancophor FFG Solution, Blancophor SV, Bodipy Fl, BOPRO 1, Brilliant Sulphoflavin FF, Calcien Blue, Calcium Green, Calcofluor RW Solution, Calcofluor White, Calcophor White ABT Solution, Calcophor White Standard Solution, Carbocyanine, Carbostyryl, Cascade Blue, Cascade Yellow, Catecholamine, Chinacrine, Coriphosphine O, Coumarin, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH—CH3, DAPI, Diamino Phenyl Oxydiazole (DAO), Dimethylamino-5-Sulphonic acid, Dipyrrometheneboron Difluoride, Diphenyl Brilliant Flavine 7GFF, Dopamine, Eosin, Erythrosin ITC, Ethidium Bromide, Euchrysin, FIF (Formaldehyde Induced Fluorescence), Flazo Orange, Fluo 3, Fluorescamine, Fura-2, Genacryl Brilliant Red B, Genacryl Brilliant Yellow 10OGF, Genacryl Pink 3G, Genacryl Yellow SGF, Gloxalic Acid, Granular Blue, Haematoporphyrin, Hoechst 33258 (bound to DNA), Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nile Red, Nitrobenzoxadidole, Noradrenaline, Nuclear Fast Red, Nuclear Yellow, Nylosan Brilliant Flavin E8G, Oregon Green, Oxazine, Oxazole, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Polyazaindacene Pontochrome Blue Black, Porphyrin, Primuline, Procion Yellow, Propidium Iodide, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Rose Bengal, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulphO Rhodamine B Can C, Sulpho Rhodamine G Extra, Tetracycline, Texas Red, Thiazine Red R, Thioflavin S, Thioflavin TCN, Thioflavin 5, Thiolyte, Thiozol Orange, Tinopol CBS, T OTO 1, TOTO 3, True Blue, Ultralite, Uranine B, Uvitex SFC, Xylene Orange, XRITC, YO PR01, or combinations thereof; and the derivatives of them. The lists of suitable fluorescent compounds/groups are also available from U.S. Pat. Nos. 6,649,414; 6,514,295; 5,073,498; 5,194,300; 4,259,313; 4,283,382 and the reference cited in these patents. Derivatives of known fluorescent compounds (such as those attached with a reactive groups e.g. an amine group or a carboxyl group) can also be used as long as they still have fluorescent property. In some embodiments, these fluorescent compounds are suitable as releasable signaling moieties for multiplexed detection as long as they can be distinguished (e.g. HPLC or CE separatable). Fluorescent nanoparticle (quantum dot) may also be used.

Signaling moieties 1 such as chemiluminescent compounds may be coupled to the carrier 2, or other components in the SAS, either permanently (non-releasable) or through a cleavable (releasable) linkage, e.g., photo-labile bond, chemical-labile bond such as an acid sensitive bond or a dissociable bond, e.g., polynucleotide base pairing. The releasable linkage includes, but is not limited to, photo labile bond, chemical sensitive bond, pH sensitive bond, and heat sensitive bond. The signaling moieties can therefore be released using a variety of methods such as oxidation, reduction, acid-labile, base labile, enzymatic, electrochemical, heat and photo labile methods, dissolution and etc. Releasable linkage may also include non-covalent bonds, which include, but are not limited to, hydrogen bonds (e.g., those in nucleic acid base pairing), ion paring, biotin-streptavidin interaction, and chelating. Under normal assay or storage conditions, the linkage between the signaling moieties 1 and carriers 2, or other component in the SAS to which the signaling moiety is bound, is stable, which permits normal assay procedures such as washing. After unbound signaling moieties are removed, bound signaling moieties 1 are cleaved or otherwise released from the SAS units with desired means. For example, one can use a UV light to cleave UV light sensitive photo labile bond that joints the signaling moieties 1 and the carriers, or other component in the SAS to which the signaling moiety is bound, thereby freeing the signaling moieties to the medium. Detection is then carried out. Release of signaling moieties from the carrier before detection can improve detection efficiency.

An analyte binding moiety can be any chemical or biological functionality with specific affinity for an analyte. They include, but are not limited to, polynucleotides, antibody, antigen, nucleic acid binding species (such as aptamers, which is nucleic acid sequence that can bind with non nucleic acid target), chelators and the like. The analyte binding moiety may be indirectly coupled to the carrier through a linker or an adaptor through, for example, a ligand-receptor binding through binding partners (e.g., biotin-avidin) or hybridization between a polynucleotide and its complementary sequence.

The carrier can be a polymer, a microparticle, or a combination of the two. Appropriate natural or synthetic polymers include, but are not limited to, oligomers (such as peptides), linear or cross-linked polymers (such as poly lysine, poly acrylic acid, proteins) or highly branched macromolecules (such as dendrimers). A chemical, biological or physical entity can be used as a carrier as long as it has one or multiple functional groups that allow direct or indirect conjugation of one or multiple numbers of signaling moieties and analyte binding moieties. The more functional groups a carrier has, the better amplification it will provide. An example of the carrier is a microparticle, which can be coated with a large number of functional groups such as carboxyl group or primary amine. Suitable size range of the microparticles includes, but is not limited to, 10 nanometers to 1000 micrometers in diameter. Suitable microparticles include, but are not limited to, microspheres, nanoparticles, liposomes, microcapsules and the like.

Preferably, the microparticle is polymer based such as varieties of micro spheres. The preferred make of microspheres is polystyrene or latex material. However, any type of polymeric make of microspheres is acceptable including but not limited to brominated polystyrene, polyacrylic acid, polyacrylonitrile, polyacrylamide, polyacrolein, polybutadiene, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethyhnethacrylate, or combinations thereof. The polymeric bead can be made easily by polymerization of monomers such as varieties of acrylates, styrenes, diene compounds or their derivatives. Suitable microspheres and the making of them are also available from U.S. Pat. Nos. 6,649,414; 6,514,295; 5,073,498; 5,194,300; 5,356,713; 4,259,313; 4,283,382 and the reference cited in these patents. Many vendors (such as Cortex biochem Inc, CA; Seradyn, Inc. IN; Dynal Biotech Inc., NY; Spherotech, Inc. IL; Bangs Laboratories, Inc. IN; Polysciences, Inc. PA) also provide suitable microspheres and micro particles and provide customer manufacture service. The microspheres can be either non cross linked or cross linked (such as contain 0.1 to 30% of a cross-linking agent, e.g. divinyl benzene, ethylene glycol dimethacrylate, trimethylol propane trimethacrylate, or N,N′ methylene-bis-acrylamide or other functionally equivalent agents known in the art). Preferably the microparticle or SAS is uniform in size and shape and preferably each microparticle contains the same or similar amount of signal groups/compounds for high sensitive detection of certain analyte. A purification step (e.g. centrifugation, filtration, size exclusion column) could be performed to purify non-uniform microparticles (e.g. those made from milling) into highly uniform micro particles.

When microparticles or the like are used as carriers, signaling moieties can be encapsulated in the particles beside coated on the surface of the particles. Encapsulation may be performed through physical means, e.g., trapping, internal adsorption, or through chemical means, e.g., covalent coupling. Alternatively, signaling moieties can first be directly or indirectly coupled to a carrier (e.g., a polymer or nanoparticles) and then encapsulated in the particle. When signaling moieties are encapsulated in particles, the particles can be dissolved, swelled, or perforated to release the signaling moieties or make them more accessible to trigger reagents. These treatments can improve the sensitivity. One could use certain physical means or certain chemicals (such as organic solvent, strong acid or base, preferably be heated) to swell or partially or completely dissolve or destroy the microparticls to release the trapped signaling moieties. For example, polymer microspheres made from monomers containing high concentration of 4-amino styrene or acrylic acid can be dissolved with acid or base respectively, similar to the method used for controlled release in pharmaceuticals. The encapsulated signaling moieties could be in the form of aggregate, e.g., small particles, powder, or crystals, which are preferably in nano meter size range. For example, when rare-earth element such as Eu is used, it could be in the form of Eu metal particles, Eu oxide particles or other Eu containing compounds aggregate. The SAS particles containing these forms of Eu or other rare earth elements are also useful for fluorescent detection or electro-chemiluminescent detection. The encapsulated rare elements can be released from the particles using physical means or certain chemicals (e.g., organic solvent, strong acid or base). Suitable chemicals for encapsulating chemiluminescent or fluorescent compounds include, but are not limited to, polymers such as polystyrene. Suitable encapsulation procedure can be found in U.S. Pat. Nos. 6,649,414; 6,514,295; 5,073,498; 5,194,300; 5,356,713; 4,259,313; 4,283,382 and the reference cited in these patents. Many vendors (such as Cortex biochem Inc, CA; Seradyn, Inc. IN; Dynal Biotech Inc., NY; Spherotech, Inc. IL; Bangs Laboratories, Inc. IN; Polysciences, Inc. PA) also provide encapsulated microspheres and encapsulation service. Signaling moieties (molecule) can be derivatized (such as attaching a lipophilic group to it) for high encapsulation rate.

Alternatively, chemiluminescent or fluorescent compound (or their derivatives) having reactive groups (such as amine group or carboxyl group) may be coupled to monomers containing reactive group (such as 4-amino styrene) and then polymerized or copolymerized to give encapsulated micro sphere. Alternatively, the microsphere can be made to have reactive group (such as amine group) inside (such as those generated from 4-amino styrene) and then couple chemiluminescent or fluorescent compound (or their derivatives, such as acridinium NHS ester) with reactive groups (such as carboxyl group) to the micro sphere. The resulting microspheres will have chemiluminescent or fluorescent compound covalently encapsulated inside. The surface of the resulting microspheres can be modified for with analyte binding groups coupling.

When used for analyte detection, the SAS can be used in a “sandwich” format or its variations. Generally, an analyte is first immobilized onto a solid phase using an analyte binding moiety such as an antibody, which preferably binds to a different epitope than the analyte binding moiety on the SAS. After wash away unbound entities, the SAS is added to a binding solution that permits the binding of SAS to immobilized analytes. After washing away unbound SAS, the bound SAS is detected by its associated signaling moieties using an appropriate instrument such as a luminometer. In certain embodiments, the mixing and binding of analyte to solid phase and SAS are performed in one step simultaneously.

In some embodiments, the SAS or the signaling moieties is separated from the solid phase capture surface (e.g., micro plate well or magnetic beads) before detection, which may reduce potential interference from the capture surface since the capture surface itself can produce significant background (e.g., background fluorescence) or the particles can block a light signal. Physical means (e.g., heat) or chemical means (e.g., appropriate acid or base, protein denaturing reagents, e.g., guanidine isothiocyanate, or the like could be used to disassociate the sandwich structure or to release the signaling moieties from the SAS, thereby separating the signaling moieties from the capture surface. If the SAS microparticle dissolution step is involved during the assay, the magnetic capture particles, if used, can be made resistant to the dissolution condition by using, for example, magnetic beads made of highly cross-linked polymer, which allows the separation of magnetic particles from SAS or signaling moieties. In microwell plate based assays, the capture surface is normally coated with analyte binding moieties, e.g., antigens or oligonucleotides, which often generate background fluorescence. In this situation, the dissociated SAS or signaling moieties or fluorescent probes are preferably transferred to a clean well for detection.

The aforementioned examples use chemiluminescent compounds or fluorescent compounds as signal moiety. The use of other types of signal moiety (molecules) in the SAS and detection techniques is also within the scope of this invention. Many detection techniques and corresponding signal molecules, which are readily available in scientific journals or textbooks (e.g. a text book for analytical chemistry) that are known to skilled in the art, are suitable for SAS based detection. Described below are a few additional examples. For example, if the detection method is mass spectrometry, the signal moieties can be any molecules as long as they can be detected by mass spectrometer. If the assay for macromolecules detection (e.g. assay to detect proteins and nucleic acids) is carried out in a well or vial, the resultant solution containing released signal molecule can be analyzed using mass spectrometry methods such as GC-MASS if the signal molecule are selected to be those detectable by GC-MS (e.g. octylamine or a lipophilic amine having higher boiling point). The use of these signal molecules make it unnecessary to use expensive MALDI or ESI Mass and allow one to measure protein and nucleic acid using any mass spectrometer as long as the signal molecules are detectable by this mass spectrometer. Examples of these molecules include, but are not limited to, alkyl amines (e.g. octylamine), many of which give strong signal in mass spectrometer and can be easily encapsulated into the microsphere carrier of SAS in large amount. Metal or metal oxide powders are also good candidates since they can be converted into many copies of metal ions for detection after being treated with acid. Under certain situations (e.g., signaling molecules are macromolecules such as the analyte binding molecules themselves), MALDI or ESI Mass is still preferred.

The detection tool is not limited to MASS spectrometry either. Any analytical technique can be used to detect the signal molecule (or its derivative) as long as the signal molecule is selected from those compatible with the analytical technique used. For example, the detection tools include chromatography methods (such as HPLC, GC, electrophoresis including capillary electrophoresis), electrical conductivity analysis, electrochemical analysis, IR, UV-visible light detection, phosphorescence analysis, luminescence analysis, colorimetric detection, radioactive detection, varieties of immunoassay, sensors, atomic spectrometer (such as atomic emission spectrometer, atomic absorption spectrometer, atomic fluorescent spectrometer), photo-acoustic method, test paper/strip and lateral flow test. The signaling molecules can also the precursor of detectable molecules.

Preferably, the signaling molecules are those molecules that give the strongest signal for the selected analytical technique. For example, the signaling molecules are preferably those that have high absorption coefficient (e.g. dyes) if UV-visible light detector is used. The signaling molecules are preferably those that have strong fluorescence (such as rare earth elements or fluorescent compounds) if fluorescence detector is used. The signaling molecules are preferably chemiluminescent agent or rare earth element if chemiluminescent detection or electro-chemiluminescent detection is used. The signaling molecules are preferably those that contain elements with high sensitivity in atomic spectroscopy if atomic spectroscopy is used. The signaling molecules are preferably those that contain high concentration of carbon if hydrogen flame detector is used. The signaling molecules are preferably those that contain high concentration of halogen or electrophilic groups (such as tetrachlorobenzene) if electron capture detector is used. The signaling molecules are preferably those molecules or elements that have high sensitivity in voltammetry or potentiometry (such as phenol or its derivatives) if voltammetry or potentiometry is used. The signaling molecule could be enzyme that can produce detectable signals or the activator or inhibitor or cofactor for certain enzyme. Some of the signaling molecules can be coupled to the SAS carrier whereas others can be encapsulated within the microparticle carrier of the SAS. Preferably, the signaling molecules are selected from those molecules that can be incorporated in the SAS in a large amount.

In some embodiments, the analyte binding molecules themselves can be the signaling molecule if they can be released and detected. The micro particle or polymer carrier themselves can also be the signaling molecules, if they can be dissolved or fragmented into many detectable small parts using certain chemical or physical means. For example, Au nanoparticle can be used as both carrier and signaling molecules since each nanoparticle contains millions of Au atoms that can be released for detection. If the signaling molecules are radioactive materials or compounds for atomic spectroscopy analysis, they may not need to be released from the SAS.

The SAS system is also suitable for inhibition (competition) binding test in addition to being used in the direct binding test. The principle of inhibition (competition) binding test is well known. Here an example is given: in order to detect analyte A in a sample, the SAS will have pre made analyte A immobilized on it instead of the analyte binding moieties specific for analyte A. Analyte A coated SAS is first incubated with the sample to be detected for the presence of analyte A. After incubation, an analyte A specific ligand, e.g., anti-A, which is coated to a solid phase (e.g., magnetic particles), is added to the reaction for further incubation. The solid phase is then removed from the solution. The analyte A in the sample, if present, competes with that on SAS, leaving behind free SAS in the solution after the removal of solid phase substrate. If there is no analyte A in the sample, SAS will bind to the solid phase substrate and be removed from the solution along with the solid phase substrate.

Alternatively, the SAS is coupled with the analyte binding molecules, (e.g., anti-A), whereas the solid phase substrate (e.g. magnetic particles) is coated with analyte A. The analyte A in the sample, if present, competes with that on the solid phase substrate, thereby blocking or reducing the binding between the solid phase substrate and SAS. The presence of analyte A in the sample again results in the presence of free SAS in the solution, which can be subsequently detected. It is understood that assay conditions (e.g., appropriate amounts of SAS) will have to be optimized.

In order to detect more than one analyte, a mixture of different SAS, each of which is labeled with distinct analyte binding moieties that target different analytes as well as signaling molecules is used. In some embodiments, the signaling molecules on different SAS type are also different.

An example of multiplexed assay using SAS is given below: For simultaneous detection of analyte A and analyte B in a sample, two SAS are prepared. One of the SAS types contains specific analyte binding moiety for analyte A and is labeled with multiple signaling molecules SA whereas the other SAS type contains specific analyte binding moiety B for analyte B and is labeled with multiple signaling molecules SB. In addition, two types of magnetic particles specific for analyte A and B, respectively, are also prepared. One can also use one type of substrate capture surface if both analyte binding moieties targeting A and B are present on the capture surface (e.g., one type of magnetic bead).

Protocols similar to those described in examples 1-4 can be employed for detecting analytes A and B simultaneously. Signaling molecules SA (if analyte A is present in the sample) and SB (if analyte B is present in the sample) can be released from corresponding SAS. Many detection tools such as those listed above can be used for detecting signaling molecules SA and SB. The signaling molecules SA and SB and corresponding detection techniques (detection tools) should be such that the two signaling molecules can be easily distinguished. For example, SA and SB can be fluorescent compounds with well-separated peak emission wavelength, or chemiluminescent compounds with distinct chemiluminescence kinetics (e.g., flash light vs. glow light), or different alkaline amines (such as octylamine and dodecyl amine) that can give distinct ion peaks on a MASS spectrometer, or different elements that are discernable with atomic spectrometry, or compounds with distinct retention time or migration patterns on HPLC or GC or CE, or compounds (e.g. different dyes) with different spectra on spectrometer (e.g. UV-Vis or IR). Several analytical techniques (e.g., LC-MS) can be combined to analyze the signaling molecules to increase the number of different types of signaling molecules and to increase the sensitivity of the assay.

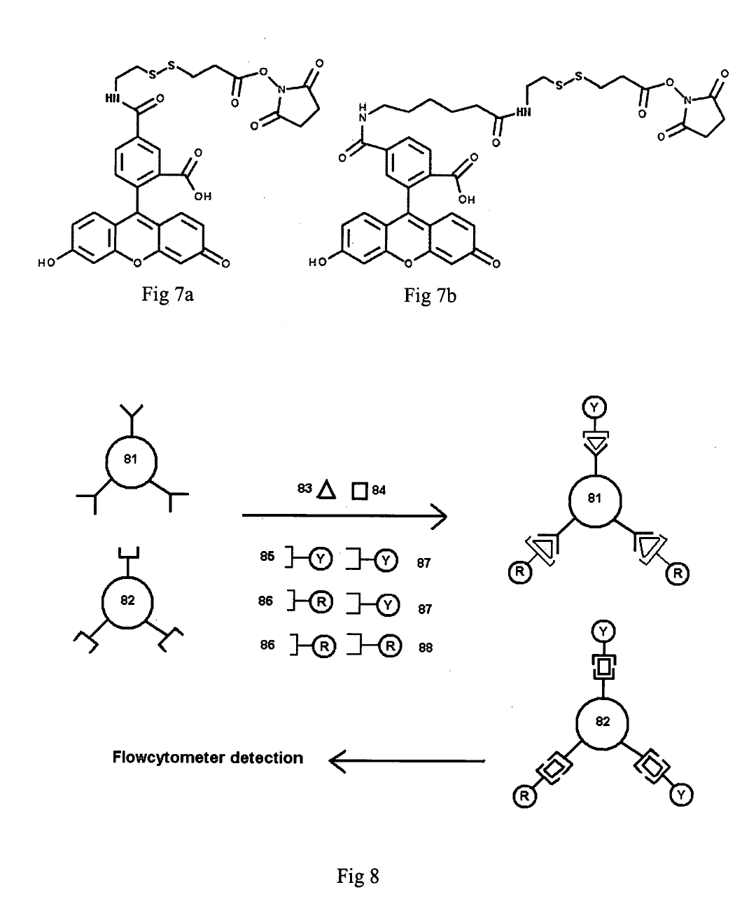

A large number of chemicals suitable for signaling molecules in SAS are commercially available from many vendors. For example, Molecular Probe (Portland, Oreg.) provides many fluorescent compounds that are suitable as signal molecules for SAS based assays. Alternatively, one can prepare appropriate compounds. For example, if a fluorescence detector is used in HPLC, a skilled in art can modify a known fluorescent molecule by, for example, attaching different length of alkyl groups to it (e.g. compounds described in FIG. 7a and FIG. 7b), thereby generating two or more fluorescent molecules that have different retention time in HPLC (e.g. reversed phase HPLC equipped with RP-18 column) due to their different length of alkyl groups. Thus, these fluorescent compounds with distinct retention time on HPLC can be used as distinct signaling molecules for the detection of multiple analytes in a multiplexed assay. The principle above is to convert one signaling molecule (e.g. chemiluminescent compounds, fluorescent compounds) into many different signaling molecules by attaching a discriminating (or separating) tag (in the above example is different alkyl groups), which can be differentiated using appropriate detection techniques.

For detecting multiple analyte using chromatography methods, the signaling molecules of the corresponding SAS preferably have different retention time or Rf value to allow the simultaneous detection of multiple analytes using these instrument, e.g., HPLC or GC or CE. Because of the amplification capability of the SAS disclosed in this invention, these multiplex assays would have vastly improved sensitivity. The amplification factor primarily depends on how many signaling molecules there are in each SAS unit. It is preferred that signaling molecules are released from the SAS carrier prior to separation and detection. The releasing of signaling molecule provides both amplification and easy identification of different analytes since different released signaling molecules can be separated and characterized easily.

It is understood that a multiplex assay disclosed here could be any assay that is designed for simultaneous detection of two or more analytes. “Simultaneous detection” here refers to a detection assay or process that requires as few as one sample input to achieve the detection of two or more analytes.

It is further understood that internal control (e.g. a positive control or a negative control) or sample loading control may be included in singular or multiplex assays. The analytes suitable for internal control or sample loading control may be artificial (e.g., an artificial nucleic acid sequence that is added to the sample prior to testing) or be always present in the sample (e.g., serum albumin in blood samples). Preferably, the analyte(s) to be detected and the analyte(s) to be used as control are similar in composition, e.g., both are nucleic acids, protein antigens or antibodies. Sample loading control or internal control indicates that sufficient sample is used for a particular test and that the testing process performs as expected. A well-defined and quantified internal control can also be used as a quantitative standard for quantitative assays. Each control is detected with an SAS specific for the control.

In some multiplex assays where the analytes are highly abundant and/or the detection method is highly sensitive and, consequently, the detection of the analytes does not require substantial signal amplification, the analyte binding moiety themselves can be the carriers, i.e., signaling molecule or molecules are attached to the analyte binding moiety. Preferably the signaling molecules are releasable from the analyte binding moiety so that they can be released prior to detection step (however if the signaling moiety-analyte binding moiety complex can still be separately detected, the signal moiety may not need to be released, e.g. fluorescent DNA probes that can be separated with HPLC or CE). Appropriate methods for generating releasable attachment are described previously in this application.

For example, in order to detect antigen A and antigen B at the same time, antibodies specific for these antigens, e.g., Ab-A1 and Ab-B1, respectively, are labeled with fluorescent signaling molecules FA and FB, respectively, through a releasable linker. Fluorescent signaling molecules FA and FB have different retention time on chromatography or different fluorescence spectra. Solid phase substrate (e.g., magnetic particles) is coated with second antibodies, Ab-A2 and Ab-B2, that are specific for antigen A and B, respectively. Ab-A2 and Ab-B2 may be used to coat the same or different magnetic particles; however, in the latter case, the two magnetic particles are mixed. A conventional sandwich type of assay may now be employed to carry out the multiplex detection. Preferably, the sample is first contacted with the substrate to allow the capture of antigens A and/or B, if present. After removal of unbound components in the sample, Ab-A1 and Ab-B1 (with fluorescent signaling molecules) are incubated with the substrate. After removal of unbound antibodies, the associated fluorescent signaling molecules are released from the antibodies. Since they have different retention time in HPLC or CE or GC, they can be detected simultaneously to indicate the presence and amount of antigen A and antigen B in the sample.

In certain embodiments, the analyte binding molecules labeled with signaling molecules can be readily differentiated on HPLC or CE. Examples include, but are not limited to, nucleic acid probes or the like that are labeled with a signaling molecules. The probes are distinct on chromatograms due to different base compositions and/or length, which result in distinct retention times. In these cases, the signaling molecules on the analyte binding molecules may not need to be released from the analyte binding molecules prior to detection. One specific example is given here: in order to simultaneously detect DNA A and DNA B in a sample, one prepares fluorescent DNA probes A1 and B1 specific for DNA 1 and DNA 2, respectively, and magnetic beads coated with capture probes A 2 and B 2 specific for DNA A and DNA B. It is preferred that the fluorescent probes and capture probes hybridize to different regions of the target nucleic acids. To perform detection, the fluorescent probes and magnetic particles are added to the sample. After heating to 94 degree for an appropriate period of time to denature the DNA target, the reaction is incubated at appropriate temperature for appropriate time to allow hybridization. After removal of unbound fluorescent probes using a magnet, the bound fluorescent probes are released from the magnetic particles using, for example, heat or alkaline condition. The released fluorescent probes are then analyzed with a chromatographic method. It may be preferred that a second complimentary to the DNA probe A1 and B1 and marked with one or more fluorescent molecules can be added before detection. The retention time of the hybrids may change but should be consistent and characteristic. This process can improve the sensitivity.

It is within the scope of this invention that the SAS is used in assays that involve the use of flow cytometer or the like instrument (e.g. a flow based detection device such as those used by Luminex liquid array system). Currently flow cytometer based assays use microspheres that are normally labeled with one or more marker (e.g., fluorescent compound) and coated with an analyte binding moiety (e.g., Ab-a) for capturing an analyte A. A second analyte binding moiety (e.g., Ab-b) specific for the same analyte (but different epitope region) is labeled with another fluorescent compound B. To perform the detection, the microspheres and second analyte binding moiety are incubated with the sample. Presence of analyte A would result in the formation of a microsphere-analyte A-fluorescent compound B complex. The reaction mix is then passed through a flow cytometer, which can detect both microspheres and fluorescent compound B. The association between microspheres and fluorescent compound B, which can be detected with the flow cytometer, indicates the presence of analyte A in the sample. Current multiplex assay format uses a mixture of several microsphere types, each of which is coated with an analyte binding moiety specific for an analyte and marked with a distinct label (e.g., fluorescent compounds) or with a distinct ratio of two fluorescent compounds. The association between second analyte binding moiety and a particular microsphere type indicates the presence of a particular type of analyte in the sample. The drawback of current flow cytometer based assays is that they have limited sensitivity.

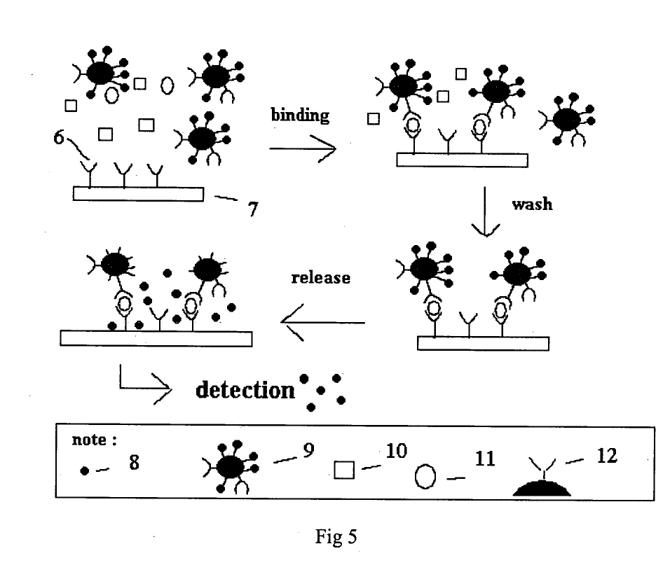

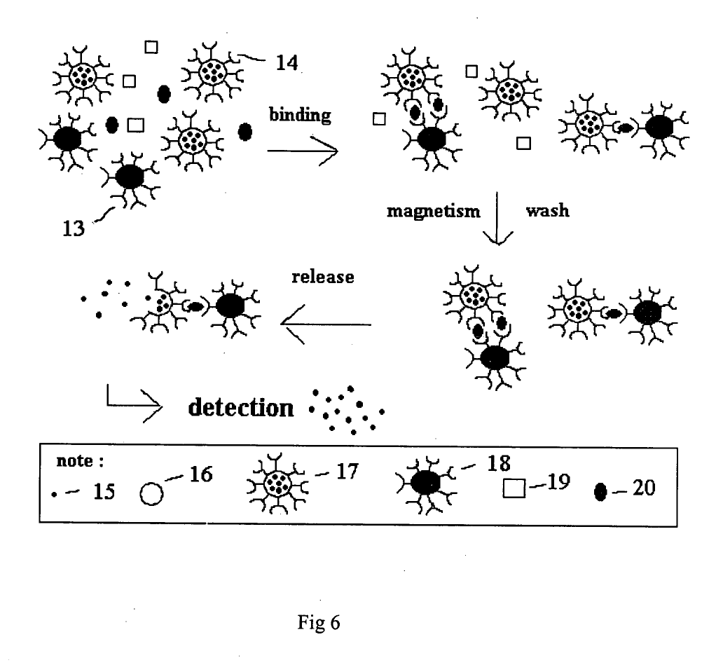

The sensitivity of flow cytometer based assays can be substantially improved according to the teaching of current invention. Refer to FIG. 4, which depicts a microparticle-based SAS, which can be used in an assay illustrated in FIG. 5 or FIG. 6. The assay depicted in FIG. 6 can be modified to be a flow cytometer based assay. The microspheres are preferably labeled with signaling moiety (e.g. fluorescent compound or compounds) and with an analyte binding moiety for specific detection of an analyte. As illustrated in FIG. 6, the presence of specific analyte results in the formation of microsphere-magnetic particle complex, which can be separated from free microspheres using a magnet. The captured microspheres are detected with a flow cytometer without releasing the labeled signaling moiety. A great number of microspheres can be captured since only a small number of analyte molecules are needed to cause microsphere-magnetic particle interaction, which greatly improves the sensitivity of the assay. The number of the captured microspheres is related to the amounts of the analyte in the sample. By counting the number of the captured microspheres using flow cytometer or the like device after separation, the concentration of analyte in the sample is determined. Therefore, the assay is quantitative as well. Capture substrate other than magnetic sphere (e.g. multi well plates) can also be used, however, they may result in lower sensitivity in some cases.

It is also within the scope of current invention that reporter system (e.g. SAS) is used in flow cytometer-based multiplex assays (e.g. example 7). Here the SAS is the microsphere. Similar to the microspheres currently used in the liquid array technology (e.g. xMAP™ technology from Luminex), several type of microspheres having different analyte binding moiety specific to different analyte are coded with specific color or bead size or color (e.g. dye, including the combination of the ratio of two or more different dyes) combination. These microspheres can be used as reporter systems to detect multiple analytes simultaneously using protocol described in the paragraph above. Since flow cytometer or the like device can count non-dyed microsphere, plain beads with no dye in the microsphere can also be used as SAS.

There are many ways to make color coded microsphere such as those disclosed in U.S. Pat. Nos. 6,649,414; 6,514,295; 5,073,498; 5,194,300; 5,356,713; 4,259,313; 4,283,382 and the reference cited in these patents. Many vendors (e.g. Cortex biochem Inc, CA; Seradyn, Inc. IN; Dynal Biotech Inc., NY; Spherotech, Inc. IL; Bangs Laboratories, Inc. IN; Polysciences, Inc. PA) also provide this kind of microspheres and microsphere manufacturing service. A skilled in the art can also use methods described in current invention. A skilled in the art may also use different dye combination (including ratio combination) to make color-coded microspheres (e.g. those for liquid array techniques or the like). A combination of two or more dyes can be used to code the beads. The dyes can have reactive groups for coupling. The dyes preferably emit fluorescent light at distinct, essentially non-overlapping wavelengths. In some embodiments, the emitted fluorescent lights are separated from one another by at least 10 nm, preferably 30 nm, and more preferably by at least 50 nm.

Another aspect of current invention relates to a method of performing multiplex detection using a flow cytometer type device and reporter systems without pre coding the microspheres with distinct color or combination of colors. In current liquid array method, microspheres having different analyte binding moiety are pre coded (e.g. using specific bead size, color and its intensity, different fluorescent dye or fluorescent dye combination, e.g. U.S. Pat. No. 6,514,295) before the assay. During an assay, different microspheres bind with different analytes, which also bind to second analyte binding molecules that have the same fluorescent label. The resulting sandwich structure, coded beads-analyte-fluorescent label, enables the identification of the beads and, consequently, the analyte or analytes present in the sample. The fluorescent label serves as an indicator for the presence of analyte or analytes in the sample whereas the codes in the beads reveal which analyte or analytes are present in the sample. Each coded bead is coupled with a specific analyte binding molecules. In current invention, instead of pre coding the microsphere with specific fluorescent dye combination, the second analyte binding molecules are coded with specific fluorescent dye combinations for the differentiation of different analytes as described in the following example. The fluorescent dyes that can be used also include fluorescent quantum dot (e.g. fluorescent nano particles). Here the reporter systems are color-coded analyte binding molecules.

For example, a combination of fluorescein, a green fluorescent compound, and rhodamine red, a red fluorescent compound are used to code the second analyte binding molecules. When associated with a microsphere or the like, both fluorescein and rhodamine red can be readily detected, differentiated and quantified in a flow cytometer. The ratios of the two dyes then can be used as codes for the second analyte binding molecules. To prepare a multiplex assay for the detection of antigens A, B, C, D, and E, it is preferred that there are available at least two specific antibodies for each antigen, e.g., Ab-A1 and Ab-A2 for antigen A. Each of the first analyte binding molecules, Ab-A1, Ab-B1, Ab-C1, Ab-D1, and Ab-E1 is coupled onto the surface of microparticles, resulting in distinct microparticles with distinct first analyte binding molecules. These microparticles are then mixed and used in the assay. Note that the microparticles are not labeled or marked with any signaling molecules (e.g. fluorescent dyes).

Each of the second analyte binding molecules, e,g, antibodies Ab-A2, Ab-B2, Ab-C2, Ab-D2, and Ab-E2 is labeled with both fluorescein and rhodamine red according to the following ratios: Ab-A2: 100% fluorescein, 0% rhodamine red; Ab-B2: 75% fluorescein, 25% rhodamine red; Ab-C2: 50% fluorescein, 50% rhodamine red; Ab-D2: 25% fluorescein, 75% rhodamine red and Ab-E2: 0% fluorescein, 100% rhodamine red. Evidently many more combinations can be achieved using more than two fluorescent dyes and more subtle combinations similar to those microspheres used in current liquid array. According to the teaching of current invention, a polymer such as protein, linear synthetic polymer or dendrimer could be used as a carrier to which the fluorescent molecules and second analyte binding molecules can be coupled. A logical extension is that the second analyte binding molecules-carrier-fluorescent molecules are nanometer-size microspheres that are encapsulated with different dye combinations and coated with specific analyte binding molecules (second analyte binding molecules) on its surface. It is understood that the ratios of the combinations may be population-based, e.g., a specified ratio is in fact the mean of a population distribution. The ratio uniformity will depend on, among other factors, relative coupling efficiency of the dyes under a coupling condition. However, so long as different second analyte binding molecules can be reproducibly differentiated by the assigned ratios, they would be appropriate to be used in a multiplex assays.

Alternatively, a second analyte binding molecules is labeled with only one dye, e.g., fluorescein or rhodamine red, but not both. A ratio is created by combining these two second analyte binding molecules with distinct dyes. For example, by combining 75% fluorescein-labeled Ab-B2 and 25% rhodamine red-labeled Ab-B2, one creates a 75% to 25% ratio. In this case, different fluorescent quantum dot (having different fluorescent spectrum) coated with analyte binding molecules can also be used to make the combination, e.g combining 75% Ab-B2-labeled quantum dotl and 25% Ab-B2-labeled quantum dot 2. Although this approach is a little more cumbersome, the ratio combinations may be better controlled.

For the detection of antigens A, B, C, D and E simultaneously, the sample are mixed with the microspheres coated with first analyte binding molecules and the second analyte binding molecules coupled with distinct ratios of fluorescein and rhodamine red. After incubation that promotes specific binding among antibodies and antigens, the reaction mix is analyzed using a flow cytometer or the like device. The presence of a particular analyte or analytes will result in the association of microspheres with a particular fluorescent ratio or ratios, which can be detected with the flow cytometer. For example, if the signal ratio of fluorescein to rhodamine red is 75% to 25% only on the microsphere, then there is only one analyte in the sample, which is antigen B. The fluorescent intensity can be measured to tell the amount of the analyte and the measured fluorescent combination will tell what analyte binding molecules is on the micropshere therefore tell the ID of the analyte.

FIG. 8 shows an example for multiplex detection of two analytes, analyte 83 and 84. The second analyte binding molecules 85, 86, 87 and 88 are color-coded (color labeled) with either Y (yellow) or R (red). Analyte binding molecules 85 (yellow coded) and 86 (red coded) are for analyte 83 whereas 87 (yellow coded) and 88 (red coded) are for analyte 84. For analyte 83, the color ratio is 1 (yellow) to 2 (red) whereas, for analyte 84, the ratio is 2 (yellow) to 1 (red). The microspheres 81 are coated with antibodies specific for analyte 83 and 82 are coated with antibodies specific for analyte 84.

To perform an assay, the sample is incubated with the microspheres and second analyte binding molecules under conditions that promote specific antigen-antibody interaction. The reaction mix is then subjected to flow cytometric detection. The detection of beads with 1:2 (yellow to red) ratio and 2:1 ratio indicate the presence of analytes 83 and 84, respectively. It is understood that the ratio may vary due to the different binding affinity of analyte binding molecules (antibodies), fluorescent intensity of dyes and etc; therefore the ratio is preferably determined experimentally. The total fluorescent intensity on each micropshere indicates the relative amounts of analytes 83 or 84 in the sample.

Although pre-color coded microspheres are not necessary required, they (e.g. the beads and methods used by BD Bioscience and Luminex) can also be used in combination with color coded second analyte binding molecules in current invention, the added coding of microspheres can further improve the confidence of ID determination or increase the detection multiplicity. The microspheres can also be purified before flow cytometric detection by means such as filtration, washing or magnetism (if using magnetic bead) to decrease the background signal caused by the unbound second probes. In some embodiments, bigger microspheres may result in higher signal per bead due to the increased binding surface. Alternatively, instead of using cytometer to detect these fluorescent beads one by one, part or entire of the microspheres population can be imaged (e.g. using a scan imaging system, a camera, preferably a CCD camera) when being irradiated to give fluorescence. The image of the fluorescent microspheres (containing their ID information and amount or fluorescence intensity) tells the ID and the a mount of the analytes in the sample. Using magnetic bead and magnetism can help immobilize the microspheres for purification and better imaging.

The following abbreviations have the meanings set forth below; Tris—Tris(hydroxymethyl)aminomethane-HCl. HPLC—high performance liquid chromatography. BSA—bovine serum albumin from Sigma Chemical Company, Mo. EDTA—ethylenediaminetetetraacetate from Sigma Chemical Company. g—grams. mM—millimolar FAM—fluorescein, pmol—pico mole; uL—micro litter. DTT—ithiothreitol, from Pierce, Ill.

### EXAMPLE 1

### Signal Amplification with Release of Signaling Moiety

FIG. 5 illustrate one example in which reporter system (e.g. SAS) is used for analyte detection. The assay is aimed to detect a certain antigen 11 in the sample containing other molecules 10, i.e., the non-target molecules. The micro well plate well surface 7 is coated with antibody 6 specific for the antigen 11 using a sandwich format method known in art. The microsphere-based SAS 9 contains another antibody 12 specific for an epitope distinct from that for antibody 6 on the microwell plate wells. In addition, SAS 9 also contains chemiluminescent molecules 8 as the signaling moieties. In the presence of antigen 11 in the sample and under appropriate binding conditions (e.g., appropriate buffer, temperature etc.), some of the SAS 9 are immobilized on the surface of the microwell plate well 7 through a sandwich binding in which the antigen interacts with both the SAS 9 and microwell plate. After washing to remove the unbound SAS 9 and analyte 11, the chemiluminescent molecules 8 are released from the SAS 9 using chemicals or light that can cleave the bonds between chemiluminescent molecules 8 and the microsphere. The released chemiluminescent molecules 8 can be readily detected using a luminometer or electro-chemiluminescence detector. Although releasing is preferred, the bound chemiluminescent compounds can also be detected without being cleaved. The chemiluminescence intensity is proportional to the amount of antigen 11 in the sample.

The chemiluminescent molecules 8 can also be replaced with fluorescent molecules as signaling moieties. The fluorescent molecules may be coupled to the particles through a linker containing a disulfide bond (e.g., 3,3′-Dithiodipropionic acid), which is thiol-cleavable. Upon binding, the unbound microspheres are removed from the well through washing. Then the fluorescent molecules are cleaved from the microspheres using a reducing agent such as beta-mercaptoethanol. The released fluorescent molecules can be readily detected using a fluorometer. The fluorescence intensity is proportional to amounts of bound microspheres, which are in turn proportional to the amount of antigen in the sample.

In certain embodiments the signaling moieties are coupled to the inside of the microparticle via cleavable linker if the microparticle is porous or solvent permeable. In other embodiments, the signaling moieties such as the chemiluminescent agents or fluorescent agents can be coupled to the inside of microparticle via non-cleavable linker since they can still give detectable signal (such as light) without being released.

### EXAMPLE 2

### Magnetic Particle Based Signal Amplification Using Reporter System for Detecting HIV RNA

FIG. 6 illustrates yet another example for using SAS in an assay. In this case, magnetic particles 18 are used as the solid phase substrate. The chemiluminescent molecules 15 are encapsulated in the microparticles 16. Magnetic particles 18 and SAS 17 are coated with distinct analyte binding moieties, e.g., polynucleotide probes 13 and 14 that hybridize with different regions of HIV-1 viral RNA 20 for the detection of this virus. The magnetic particles are preferably approximately 3 micrometer in diameter and are coated with functional groups such as carboxyl group, which facilitates the labeling of analyte binding moieties such as oligonucleotide probe 13. An example for suitable magnetic particles is Dynabeads M-270 coated with carboxylic acid (available from Dynal Biotech, Oslo, Norway). Dynal Biotech provides a protocol for labeling of oligonucleotides to the magnetic particles.

An assay requires at least one set of probes, e.g., polynucleotide probes 13 and 14, although more than one set of probes is preferred since more probes may provide stronger binding. The probes may not necessarily need to be conjugated to magnetic particles or SAS unit if the probes also contain a suitable binding partner, e.g., biotin, or a specific nucleic acid sequence, which can be used for binding to magnetic particles or SAS units through ligand-receptor binding or nucleic acid hybridization.

The sample to be tested is first treated with appropriate reagents and conditions, e.g., a buffer containing guanidine thiocyanate, to denature the proteins and release the nucleic acids in the sample. Magnetic particles 18 are added and incubated for an appropriate period time (e.g., 60 minutes) at appropriate temperature (e.g., 50 degree C.) in an appropriate buffer (e.g., 0.1 M PBS buffer) that promotes nucleotide hybridization. The capture magnetic particles 18 are then washed several times to remove unbound entities 19, suspended in an appropriate buffer that promotes specific hybridization, and then incubated with SAS microparticles 17. If HIV-1 RNA 20, or an appropriate portion of it, is present, the magnetic particles 18 and SAS microparticles 17 will be bound together through HIV-1 RNA 20. After washing away unbound SAS microparticles using a magnet or its equivalent, the bound SAS microparticles are dissolved with an appropriate solvent, e.g., dimethylsulfoxide (DMSO) for the polystyrene particles, and released chemiluminescent compounds 15 are detected with an instrument such as luminometer.

Because only a few analyte molecules are needed to provide stable binding between magnetic particles 18 and SAS particles 17 and because each SAS microparticle 17 is encapsulated with a large number of chemiluminescent molecules 15, the signal is greatly amplified. The sensitivity of the assay depends on several factors, including the minimal number of SAS microparticles 17 that can be detected and the efficiency of removing unbound SAS microparticles 17 (the background). For example, if the minimal number of SAS microparticles 17 that can be detected is ten (10) and all unbound SAS particles 17 are removed, then the sensitivity of the assay is ten HIV-1 RNA copies. When the magnetic particles 18 and polystyrene microparticle based SAS 17 labeled with acridinium as chemiluminescent molecule are used for detecting HIV-1 viral RNA 20, as low as ten copies of virus can be detected. To increase the stability of the magnetic particles 18-HIV-1 RNA 20-SAS 17 complex, multiple pairs of probes are used with each pair hybridizing to different regions of the HIV-1 RNA 20. Preferably, but not necessarily, one probe in a pair is coupled to magnetic particle 18 whereas the other probe in a pair is hybridized to the SAS 17. The use of multiple different probes on each magnetic particles 18 and SAS unit 17 can improve the sensitivity for HIV-1 RNA detection. In this case even one copy of HIV-1 RNA 20 could result in multiple polynucleotide binding pairs between the HIV-1 RNA 20 and magnetic particles 18 and between the HIV-1 RNA 20 and the SAS 17.

### EXAMPLE 3

### Detection of Bacteria

This example shows how the SAS technology can be used for sensitive detection of a particular species or class of species of bacteria using a nucleic acid target, e.g., tRNA, ribosomal RNA. Similar to the HIV-1 assay, there needs to be at least one pair of probes. Here in this example, the probes are relatively long oligonucleotides that contain two hybridization domains, one of which is specific for the target nucleic acids whereas the other domain is specific for the oligonucleotides conjugated on magnetic particles for one of the probes or for the oligonucleotides conjugated on SAS units for another probe. In this example, we use Probes A and B, which contain hybridization domains for magnetic particles and SAS, respectively. The magnetic particles are preferably approximately 3 micrometer in diameter and are coated with functional groups such as carboxyl group, which facilitates the labeling of analyte binding moieties such as oligonucleotide probe. An example for suitable magnetic particles is Dynabeads M-270 coated with carboxylic acid (available from Dynal Biotech, Oslo, Norway).

Teachings for preparing various components for the assay were described in U.S. provisional patent application 60/555,683, and U.S. patent application Ser. No. 10/205,195, each incorporated here by reference.

The sample to be tested is mixed with 2 volumes of appropriate lysis buffer, e.g., 2 mL 50 mM Tris-HCl, pH 7.4, 5 M guanidine thiocyanate and 2% Triton X-100, and rotated at room temperature for 20 minutes. Appropriate amounts of at least one pair of probes, e.g. 10⁹ copies in 1 mL PBS buffer, which hybridize to different regions of the target RNA, are added to the lysed sample. The reaction mix is heated to 94 degree for 5 minutes and then incubated at 50 degree for 15 to 60 minutes to allow the probes to anneal to the target nucleic acids, which results in the formation of Probe A-Target RNA-Probe B complex. After addition of an appropriate amount of magnetic particles, e.g., 10⁷ particles, the reaction mix is incubated for 15 to 60 minutes with agitation to allow the hybridization of all Probe A, which contains the hybridization domain for the polynucleotide probe on the magnetic particles. The reaction solution is then removed using a magnet. The magnetic particles are washed three times with 1 to 2 mL of washing buffer, e.g., phosphate saline buffer (PBS). If there is a sufficient amount of target nucleic acids in the sample, the magnetic particles will be labeled with Probe B through its binding to target nucleic acids.

To detect Probe B bound to the magnetic particles, the washed magnetic particles are suspended in 100 microliters hybridization buffer, e.g., PBS with 10 mM aminoethanethiol, 2% Tween 20. After addition of appropriate amounts of SAS, e.g., 10⁶ particles, which is conjugated with an oligonucleotide that can hybridize with Probe B, the mix is incubated for 15 to 60 minutes under appropriate conditions that promote specific hybridization. The magnetic particles are then washed to remove unbound SAS. The bound SAS is detected through an appropriate instrument such as a luminometer. Alternatively, the bound SAS can be released from magnetic particle by mixing with 1 mL 0.1 N HCL for 3 minutes before detection, which can enhance the efficiency of signal detection.

### EXAMPLE 4

### Encapsulation of Acridinium in a Microparticle

This example teaches a method for encapsulating acridinium, or its derivatives, into microparticles that can be used as part of the SAS. Because of the charge present in a typical acridinium molecule, a sufficiently hydrophobic moiety or moieties are preferably attached to acridinium, thereby creating an acridinium derivative that is sufficiently hydrophobic, i.e., substantially insoluble in aqueous solution. Such a hydrophobic acridinium species minimizes the leaching from inside the microparticles under aqueous conditions once it is encapsulated inside the microparticles.

Hydrophobic acridinium derivatives are normally dissolved in an organic solvent so that a concentrated acridinium solution can be created for encapsulation. The microparticles used for encapsulation should be compatible with the organic solvent, i.e., the basic structure of microparticles should not be partially or completely dissolved, or otherwise significantly altered. For example, polystyrene-based microparticles may be dissolved in certain organic solvents such as chloroform, but would be compatible with the others such as ethanol. Polystyrene microparticles copolymerized with a cross-linking polymer(s) may be compatible with most organic solvents, including dichloromethane and chloroform. The microparticles are preferably, but not necessarily, functionalized with functional groups such as primary amine or carboxyl group. The following procedure teaches one method for encapsulating acridinium or its derivatives in microparticles. It is understood that different methods, or variations of the current method, can also be used to achieve sufficient encapsulation of acridinium or its derivatives.

This procedure teaches the encapsulation of an acridinium derivative, 4-dodecylphenyl-10-methylacridinum-9-carboxylate trifluoromethane sulfonate, in a cross-linked polystyrene microparticles (Spherotech, Inc, catalog number APX-20-10, 2.48 micrometer in diameter), which is functionalized with primary amines. Prior to encapsulation, the microparticles are washed twice with dry ethanol (100%) and then twice with acetone. One hundred milligrams of the acridinium derivative is dissolved in 1. 0 mL CH₂Cl₂ (dichloromethane) to make 10% (100 mg/mL) solution, which is used to suspend 50 mg cross linked polystyrene particles prepared as described above. The microparticle solution is then rotated in a rotating device for 30 minutes at room temperature. The microparticles are then filtrated through a 0.1 micrometer VVPP filter (Millipore catalog number VVLP04700) and washed with 10 mL of 50% ethanol aqueous solution three times. The microparticles are then dialyzed in 1 LPBS buffer ovenight to remove unencapsulated acridinium. The dialysis step is repeated once or more. The resulting microparticles are now encapsulated with the acridinium derivative. The functional groups on the microparticles surface can be used to directly or indirectly couple with analyte binding moieties. The resultant SAS can be used for analyte detection. It is preferred, but not necessary, to release the encapsulated acridinium derivative prior to chemiluminescent detection during an assay by incubating for two minutes with an organic solvent such as DMSO.

Reagents for Examples 5-8:

Target 1:
5′ - AGT TGG TAG AGC ACG ACC TTG AGT TCG AGT CTC
GTT TCC C-3′
Target 2:
5′- ACA CAA CTG TGT TCA CTA GCG TTG AAC GTG GAT
GAA GTT G-3′
Capture probe1:
5′-/5Bio/AAA AAA GGG AAA CGA GAC TCG AAC TC-3′
Capture probe 2:
5′-/5Bio/AAA AAA CAA CTT CAT CCA CGT TCA A -3′
Signal probe 1F:
5′- AAG GTC GTG CTC TAC CAA CTA AA/36-FAM/-3′
Signal probe 2F:
5′-GCT AGT GAA CAC AGT TGT GTA AAA AAA/36-FAM/-3′
Signal probe 1bio:
5′- AAG GTC GTG CTC TAC CAA CTA AA/3Bio/-3′
Signal probe 2bio:
5′- GCT AGT GAA CAC AGT TGT GTA AAA AAA/3Bio/-3′
Signal probe 1am:
5′- AAG GTC GTG CTC TAC CAA CTA AA/3AmMC7/-3′
Signal probe 2am:
5′-GCT AGT GAA CAC AGT TGT GTA AAA AAA/3AmMC7/-3′
Signal probe 1c3:
5′- AAG GTC GTG CTC TAC CAA CTA AA/3Cy3Sp/-3′
Signal probe 1c5:
5′- AAG GTC GTG CTC TAC CAA CTA AA/3Cy5Sp/-3′
Signal probe 2c3:
5′-GCT AGT GAA CAC AGT TGT GTA AAA AAA/3Cy3Sp/-3′
Signal probe 2c5:
5′-GCT AGT GAA CAC AGT TGT GTA AAA AAA/3Cy5Sp/-3′

The above oligonucleotides are from Integrated DNA Technologies, Inc., IA. Each oligonucleotide is dissolved in 0.1 M PBS buffer at the concentration of 1 pmol/uL.

### EXAMPLE 5

### Multiplexed Analyte Detection with Fluorescent DNA Probes as Reporter Systems

Assays: In this example, two target molecules (Target 1 and Target 2) are simultaneously detected. Micro well plate coated with DNA is used as capture substrate. The Nunc Immobilizer Streptavidin plate (Nalge Nunc International, NY) is washed with 0.1M PBS buffer three times. 50 uL capture probe 1 solution and 50 uL capture probe 2 solution are added to each well of the plate. The plate is incubated with gentle agitation for one hour at room temperature and then washed with tween buffer (0.05% TWEEN 20 in 0.1 M PBS) 3×300 uL/well followed by 0.1 M PBS buffer 3×300 uL/well. Next both target molecules (20 uL Target 1 and 20 uL Target 2 solution/well) are added followed by the addition of both fluorescent DNA probes (30 uL Signal probe 1F and 30 uL Signal probe 2F solution/well) as reporter systems. The plate is incubated with gentle agitation for one hour at room temperature and then washed with tween buffer (0.05% TWEEN 20 in 0.1 M PBS) 3×300 uL/well followed by 0.1 M PBS buffer 3×300 uL/well. The release of bound fluorescent DNA probes is archived by addition of 100 uL 0.05 N NaOH/well and incubation for 2 minutes. The released signal probes are neutralized to pH=7 with 1M PBS buffer.

Detection: The neutralized signal probes solution is analyzed with HPLC equipped with fluorescent detector (Ex./Em.: 488 nm/520 nm). The HPLC condition is RP-18 reversed phase column and the gradient for mobile phase is from 5% buffer B to 95% buffer B in 40 minutes (Buffer A: 100 mM Trimethylammonium acetate pH 7.0; Buffer B: Acetonitrile). Alternatively, an ion exchange column can be used (e.g. Hamilton PRP—X600 anion exchange HPLC column); the gradient is: A) 20 mM TRIS, 1 mM EDTA pH 9.0; B) 1N Sodium Chloride in 20 mM TRIS, 1 mM EDTA, 5-95% B (0-45 min). The corresponding peaks of these two probes showed in HPLC spectral indicate the presence of both analyte targets. Because the two signal probes have different charge/mass ratio, they can also be separated and detected with CE equipped with fluorescent detector. However, capillary electrophoresis has low sample loading, typically in several nanolitter range, which may result in low detection sensitivity compared with HPLC detection. HPLC has much higher sample loading volume, e.g. 0.5 ml can be easily achieved, which is half million times higher than the typical loading of capillary electrophoresis, and in turn can result in half million times higher sensitivity theoretically. The molecular weight of signal probe 1F is 7586 and 8934 for signal probe 2F, therefore the presence of corresponding peaks in mass spectrometry (e.g. ESI or MALDI) a lso indicate the presence of the two analyte targets.

### EXAMPLE 6

### Multiplexed Analyte Detection with Release of Signaling Moiety

In this example, the assay conditions are identical to those described in example 5 except the different reporter systems are used. The two reporter systems are made by coupling a fluorescein NHS ester derivative containing an disulfide bond (FIG. 7a) with signal probe 1 am and a fluorescein NHS ester derivative containing an disulfide bond and an additional hexanoyl moiety (FIG. 7b) with signal probe 2 am. The NHS ester group can react with the amine group on signal probe lam or 2 am to form a stable amide bond. The disulfide bond can be cleaved to release the corresponding fluorescein derivative with certain chemicals. Therefore these, two fluorescent probes are used as reporter systems having releasable signaling moieties.

The assay is carried out as described in example 5. However, instead of adding NaOH aqueous solution, 100 uL 50 mM DTT is added to the well to cleave the disulfide bond for 10 minutes. The resulting solution is collected and analyzed with reversed phase HPLC using conditions descried in example 5. Two peaks shown in HPLC indicate the presence of both analyte. The hexanoyl moiety containing released signaling moiety is more hydrophilic therefore has longer retention time.

### EXAMPLE 7

### Multiplexed Analyte Detection with Color Coded Microspheres as Reporter System Using Flowcytometer

In this example, the assay conditions are identical to those described in example 5 except color coded microspheres are used as reporter systems. Two color coded microspheres are used. One is streptavidin fluorescent Nile Red particle (SVFP-0556-5, 0.6 micrometer in size) coated with signal probe 1 bio for the detection of target 1. Another is avidin fluorescent yellow particle (VFP-2052-5, 1.8 micrometer in size) coated with signal probe 2 bio for the detection of target 2. The particles are from Spherotech, Inc. IL and the coating of oligonucleotides are performed according the vendor'sprotocol. Because these two microspheres are labeled with fluorescent yellow and fluorescent Nile Red that have well separated EM/EX spectral; therefore they can be identified easily by suitable flowcytometer that can distinguish them.

After the assay, the release of bound color coded microspheres from substrate is archived by addition of 0.1 N HCL and incubation for 3 minutes. The released microspheres is neutralized to pH=7 and analyzed with a flowcytometer. The presence of the two well separated peaks indicate the presence of the two types of microspheres, which in turn indicate the presence of the two analyte targets. Alternatively, the two microspheres can be distinguished by their size using a flowcytometer that can discriminate particle size (e.g. a NPE Quanta bench top cell analyzer, NPE Systems, Inc. FL). Although in current example the microspheres are coded and distinguished with two pure distinct color, more complicated color coding schemes (e.g. by different color intensity and/or color combinations, e.g. SPHEROTM Flow Cytometry Multiplex Bead Assay Particles or the beads for BD Biosciences'Cytometric Bead Array or beads for Luminex'sliquid array) as well as in combination with size coding can also be utilized to expand the detection multiplicity.

Yet another variation of this example is the two fluorescent microspheres used as reporter system are encapsulated with different fluorescent dyes having different lipophilicity. After the assay, the dyes are released from the microsphere with organic solvent and distinguished with HPLC equipped with fluorescent detector.

### EXAMPLE 8

### Multiplexed Analyte Detection with Color Coded Analyte Binding Groups as Reporter System Using Flowcytometer

In this example color coded analyte binding groups are used as reporter systems. The color coded analyte binding groups for target 1 is the mixture of fluorescent nucleic acid probes having a cy3:cy5 ratio of 3:1. The color coded analyte binding groups for target 2 is the mixture of fluorescent nucleic acid probes having a cy3:cy5 ratio of 1:3. Streptavidin coated polystyrene particles (SVP-50-5, 5 micrometer in size from Spherotech, Inc. IL) are used as capture substrate. The particles are divided into two groups. Group 1 is further coated with capture probe 1 and group 2 is coated with capture probe 2. Excess probe 1 and 2 are removed by centrifugation or filtration. To perform the assay, 100000 particles from group 1 and 100000 particles from group 2 are mixed in 100 uL 0.1 M PBS. Both target molecules (10 uL Target 1 and 10 uL Target 2 solution) are also added. The reaction is incubated with gentle agitation for half hour at room temperature and then the microspheres are washed with tween buffer (0.05% TWEEN 20 in 0.1 M PBS) 2×1 mL followed by 0.1 M PBS buffer 2×1 mL. Then followed by the addition of fluorescent DNA probes as reporter systems (90 uL Signal probe 1c3, 30 uL Signal probe 1c5, 30 uL Signal probe 2c3, 90 uL Signal probe 2c5) as reporter systems. The reaction is incubated with gentle agitation for half hour at room temperature and then the microspheres are washed with tween buffer (0.05% TWEEN 20 in 0.1 M PBS) 2×1 mL followed by 0.1 M PBS buffer 2×1 mL.

The resulting microspheres is resuspended in 200 uL 0.1M PBS buffer and analyzed with a flowcytometer. Because the Cy3 and Cy5 ratio on particles of group 1 is different with Cy3 and Cy5 ratio on particles of group 2 after the assay and Cy3 and Cy5 has well separated EM/EX wavelength, the particles from each group is easily distinguished in flowcytometer and therefore indicate the presence of analyte targets. In some embodiment, the washing step in the assay is not required and the two incubation steps can be combined.

From the foregoing it will be appreciated that, although specific embodiments of the invention have been described herein for purposes of illustration, various modifications may be made without deviating from the spirit and scope of the invention. Accordingly, the invention is not limited except as by the following claims.

1. A reporter system set for multiplexed analyte detection in a sample, comprising a group of different reporter systems each containing an analyte binding moiety specific to one analyte type and a signaling moiety.

2. The reporter system set of claim 1 wherein the different reporter systems can be distinguished with a detection tool selected from HPLC, CE, Mass spectrometer and flowcytometer type device.

3. The reporter system set of claim 2 wherein the different reporter systems are fluorescent analyte binding molecules that can be distinguished with a detection tool selected from HPLC, CE and Mass spectrometer.

4. The reporter system set of claim 2 wherein the different reporter systems are color coded analyte binding molecules that can be distinguished with flowcytometer type device when they bind to microspheres.

5. The reporter system set of claim 2 wherein the different reporter systems are different particles that can be distinguished with flowcytometer type device.

6. The reporter system set of claim 1 wherein the signaling moiety is releasable from the reporter system and the released signaling moieties from different reporter systems are different and can be distinguished with a detection tool.

7. The reporter system set of claim 6 wherein the detection tool is selected from HPLC, CE, GC, Mass spectrometer and atomic spectrometer.

8. The reporter system set of claim 6 wherein the released signaling moiety is selected from fluorescent molecules, chemiluminescent molecules, dyes, molecules having different molecular weights, electrochemiluminescent molecules, electrochemical reactive molecules and molecules containing different elements.

9. The reporter system set of claim 1 wherein the reporter systems are particles containing an analyte binding moiety and a signaling moiety.

10. A method of analyzing a sample for multiplexed analyte detection, comprising
contacting the sample with a set of first analyte binding moieties associated with a substrate to form a bound complex on the substrate;
contacting the bound complex with a reporter system set of claim 1;
separating reporter systems that do not bind the analyte and retaining reporter systems that do bind the analyte on the substrate;
distinguishing different retained reporter systems or part of them with a detection tool.

11. The method of claim 10 wherein the substrate is a particle.

12. The method of claim 11 wherein the particle is a magnetic particle.

13. The method of claim 10 wherein the substrate is micro well plate.

14. The method of claim 10 wherein the reporter systems are different particles that can be distinguished with a detection tool.

15. The method of claim 14 wherein the detection tool is a flowcytometer type device.

16. The method of claim 14 wherein different particles are color coded.

17. The method of claim 14 wherein different particles are size coded.

18. The method of claim 10 wherein the signaling moieties of the reporter systems are releasable and can be distinguished with a detection tool.

19. The method of claim 18 wherein the detection tool is selected from HPLC, CE, GC, Mass spectrometry and atomic spectrometer.

20. The method of claim 18 wherein the released signaling moiety is selected from fluorescent molecules, chemiluminescent molecules, dyes, molecules having different molecular weights, electrochemiluminescent molcules, electrochemical reactive molecules and molecules containing different elements.

21. A method of analyzing a sample for multiplexed analyte detection, comprising
contacting the sample with a set of first analyte binding moieties associated with particles to form a bound complex on the particles;
contacting the bound complex with a reporter system set of claim 1;
distinguishing different reporter systems bound particles with a detection tool.

22. The method of claim 21 wherein the reporter systems are color coded analyte binding molecules and the detection tool is a flowcytometer type device.

23. The method of claim 21 wherein a washing step is performed to remove unbound reporter systems before distinguishing the particles.
